(19) United States
(12) Patent Application Publication
(10) Pub. No.: US 2018/0335431 A1
(43) Pub. Date: Nov. 22, 2018

(54) NOVEL MULTIPLEX ASSAYS TO DIAGNOSE OR EVALUATE DISEASES OR DISORDERS IN MAMMALS

(71) Applicant: THE REGENTS OF THE UNIVERSITY OF COLORADO A BODY CORPORATE, DENVER, CO (US)

(72) Inventors: LIPING YU, CENTENNIAL, CO (US); MARIAN REWERS, DENVER, CO (US); AARON MICHELS, CENTENNIAL, CO (US)

(21) Appl. No.: 15/777,105

(22) PCT Filed: Nov. 17, 2016
(86) PCT No.: PCT/US16/62469

§ 371 Date: May 17, 2018

Related U.S. Application Data
(60) Provisional application No. 62/256,624, filed on Nov. 17, 2015.

(51) Int. Cl. G01N 33/564

(52) U.S. Cl. CPC G01N 33/564; G01N 2800/24

(57) ABSTRACT

The invention includes methods and kits that can be used to simultaneously determine whether an antibody out of a plurality of antibodies is present in a sample. In certain embodiments, at least one of the antibodies comprises an autoantibody. In other embodiments, the methods and kits of the invention can be used to simultaneously determine if a subject is likely to develop or has developed each one of a plurality of diseases or disorders.

### CROSS-REFERENCE TO RELATED APPLICATIONS

The present application claims priority under 35 U.S.C. § 119(e) to U.S. Provisional Application No. 62/256,624, filed Nov. 17, 2015, which application is incorporated herein by reference in its entirety.

### STATEMENT REGARDING FEDERALLY SPONSORED RESEARCH OR DEVELOPMENT

This invention was made with government support under DK32083 awarded by National Institutes of Health. The government has certain rights in the invention.

### BACKGROUND OF THE INVENTION

An autoantibody is an antibody produced by a mammal's immune system and directed against one or more of the mammal's own proteins. Many autoimmune diseases (notably lupus erythematosus) are caused by such autoantibodies. Normally, the immune system is able to recognize and ignore the body's own healthy proteins, cells, and tissues, not overreacting to non-threatening substances in the environment, such as foods. However, the immune system may at times cease to recognize one or more of the body's normal constituents as “self,” leading to production of pathological autoantibodies. These autoantibodies attack the body's own healthy cells, tissues, and/or organs, causing inflammation and damage. Autoantibodies may also play a nonpathological role. For instance, they may help the body destroy cancers and eliminate waste products. Autoantibodies may also play a role in normal immune function.

The causes of autoantibody production are varied and not well understood. Some autoantibody production is due to a genetic predisposition combined with environmental triggers (e.g., a viral illness or prolonged exposure to certain toxic chemicals). While families may be susceptible to autoimmune conditions, individual family members may have different autoimmune disorders, or may never develop an autoimmune condition.

The incidence of type 1 diabetes (T1D), the immune-mediated form of diabetes, has doubled in the last 20 years, especially in young children. T1D affects an estimated 1.4 million people in the U.S. alone, with an equal number of people with preclinical disease characterized by multiple islet autoantibodies (iAbs), but still normal glucose homeostasis. The presence of iAbs, their number and levels, are currently used to evaluate diabetes risk (or diabetes development stage) and as inclusion criteria into prevention trials. Nearly all children positive for iAbs against two or more targets selected from insulin (IAA), glutamic acid decarboxylase (GADA), islet antigen 2 (IA-2A) and zinc transporter 8 (ZnT8A) develop clinical T1D. When identified prior to the onset of symptoms, these children can avoid life-threatening diabetic ketoacidosis and hospitalization, as well as participate in trials to prevent T1D or studies to define the causes of T1D.

In addition, one third of the patients present with autoimmune thyroiditis or celiac autoimmunity at diagnosis of T1D. The prevalence of celiac disease is ˜1:100 in Europe and North America. However, most patients are undiagnosed or diagnosed with significant delay. Gluten-free diet is an effective treatment, and early detection by measuring transglutaminase autoantibodies (TGA) has been widely recommended. Celiac disease and T1D share HLA Class II and non-HLA genetic susceptibility and co-occur in up to 10% of the patients. The American Diabetes Association recommends routine screening for celiac disease at the time of T1D diagnosis. Combined population screening for pre-clinical T1D and celiac disease would be a rational pairing. Preventive trials for T1D are underway and likely to expand to multiple candidate interventions; however, mass screening for eligible subjects remains a laborious bottleneck.

There is thus a need in the art for novel methods of identifying patients who are at risk of developing, or have developed, autoimmune diseases or disorders. Availability of a multiplex autoantibody assay could greatly simplify diagnosis of T1D and screening for the associated autoimmune conditions. The present invention addresses and meets this need.

### BRIEF SUMMARY OF THE INVENTION

The invention provides a method of simultaneously detecting the presence or absence of each one of a plurality of antibodies in a sample. The invention further provides a method of simultaneously determining if a mammal is likely to develop or has developed each one of a plurality of diseases or disorders, wherein each one of the plurality of diseases or disorders is characterized by the presence of at least one autoantibody in a biological sample of the mammal. The invention further provides a kit for determining if a mammal is likely to develop or has developed each one of a plurality of diseases or disorders, wherein each one of the plurality of diseases or disorders is characterized by the presence of at least one autoantibody in a biological sample of the mammal.

In certain embodiments, the method comprises the steps of: (1) contacting a volume of the sample with an antigen for each one of the plurality of antibodies, wherein each antigen comprises the antigen derivatized with a detectable label (first derivatized antigen) and the antigen derivatized with a tagging label (second derivatized antigen), thus forming a second solution, wherein, if a given antibody selected from the plurality of antibodies is present in the sample, a corresponding first derivatized antigen-given antibody-second derivatized antigen complex is formed in the second solution; (2) contacting the second solution with a solid surface comprising a plurality of non-overlapping areas, wherein each one of the plurality of non-overlapping areas is derivatized with a capture molecule that binds specifically to one of the tagging labels, wherein each one of the tagging labels does not bind with more than one of the plurality of non-overlapping areas, thereby immobilizing each of the first derivatized antigen-given antibody-second derivatized antigen complexes, if present in the second solution, in a different non-overlapping area (which is thereby known to be associated with the given antibody); and (3) detecting the presence or absence of each one of the first derivatized antigen-given antibody-second derivatized antigen complexes by determining presence or absence of the detectable label immobilized to the non-overlapping area associated with the given antibody.

In certain embodiments, at least one of the plurality of antibodies comprise an autoantibody. In other embodiments, each one of the plurality of antibodies comprises an autoantibody.

In certain embodiments, the sample comprises a biological sample from a mammal. In other embodiments, the biological sample from the mammal comprises at least one selected from the group consisting of urine, blood, serum, plasma and saliva.

In certain embodiments, the detectable label comprises an electrochemiluminescence (ECL) label. In other embodiments, the ECL label comprises a ruthenium complex. In yet other embodiments, the ECL label comprises [Ru(BPy)₃]²⁺.

In certain embodiments, step (1) comprises, independently for each one of the plurality of antibodies, one of the following steps: (a) contacting the sample with an antigen for one of the plurality of antibodies, wherein the antigen comprises the antigen derivatized with a detectable label, thus forming a first solution; and subsequently contacting the first solution with an antigen for the same antibody, wherein the antigen comprises the antigen derivatized with a tagging label; (b) contacting the sample with an antigen for one of the plurality of antibodies, wherein the antigen comprises the antigen derivatized with a tagging label, thus forming a first solution; and subsequently contacting the first solution with an antigen for the same antibody, wherein the antigen comprises the antigen derivatized with a detectable label; and (c) contacting the sample with an antigen for one of the plurality of antibodies, wherein the antigen comprises the antigen derivatized with a tagging label and the antigen derivatized with a detectable label.

In certain embodiments, the tagging label comprises at least one selected from the group consisting of biotin, carbohydrate, and immunoglobulin or fragment thereof. In other embodiments, the capture molecule comprises at least one selected from the group consisting of avidin, streptavidin, lectin, protein A/G, and an aptamer. In yet other embodiments, the plurality of antibodies comprises at least four antibodies. In yet other embodiments, the plurality of antibodies comprises at least six antibodies. In yet other embodiments, the plurality of antibodies comprises at least eight antibodies. In yet other embodiments, the plurality of antibodies comprises at least ten antibodies. In yet other embodiments, the volume of the sample ranges from about 6 μL to about 20 μL, about 6 μL to about 18 μL, about 6 μL to about 16 μL, about 6 μL to about 14 μL, about 6 μL to about 12 μL, about 6 μL to about 10 μL, or about 6 μL to about 8 μL.

In certain embodiments, the plurality of antibodies comprises at least one selected from the group consisting of IAA (insulin autoantibody), GADA (glutamic acid decarboxylase autoantibody), IA-2A (islet antigen 2 autoantibody), TGA (transglutaminase autoantibody), TPOA (thyroperoxidase autoantibody), ThgA (thyroglobulin autoantibody), IFNαA (interferon alpha autoantibody), ZnT8A (zinc transporter type 8 autoantibody), 21-hydroxylase autoantibody, Cyclic Citrullinated Peptide (CCP) autoantibody, anti-double stranded DNA (dsDNA) autoantibody, antinuclear antibodies (ANA), ATPase autoantibody, and anti-myelin basic protein (MBP) autoantibody. In other embodiments, the plurality of antibodies comprises IAA, GADA, IA-2A, TGA, TPOA, ThgA, IFNαA and ZnT8A.

In certain embodiments, the mammal is human.

In certain embodiments, the method comprises: (1) contacting the sample with a plurality of antigens, wherein each of the plurality of antigens specifically, and exclusively from each other, binds to one autoantibody associated with one of the plurality of diseases or disorders, wherein each antigen comprises the antigen derivatized with a detectable label (first derivatized antigen) and the antigen derivatized with a tagging label (second derivatized antigen), thus forming a second solution, wherein, if a given autoantibody associated with one of the plurality of diseases or disorders is present in the sample, a corresponding first derivatized antigen-given autoantibody-second derivatized antigen complex is formed; (2) contacting the second solution with a solid surface comprising a plurality of non-overlapping areas, wherein each one of the plurality of non-overlapping areas is derivatized with a capture molecule that binds specifically to one of the tagging labels, wherein each one of the tagging labels does not bind with more than one of the plurality of non-overlapping areas, thereby immobilizing each of the first derivatized antigen-given autoantibody-second derivatized antigen complexes, if present in the second solution, in a different non-overlapping area (which is thereby known to be associated with the given autoantibody); and (3) detecting the presence or absence of each one of the first derivatized antigen-given autoantibody-second derivatized antigen complexes by determining presence or absence of the detectable label in the non-overlapping area associated with the given autoantibody; wherein the presence of a first derivatized antigen-given autoantibody-second derivatized antigen in the non-overlapping area associated with the autoantibody indicates that the mammal is likely to develop or has developed the disease or disorder associated with the autoantibody.

In certain embodiments, the disease or disorder comprises an autoimmune disease. In other embodiments, the disease or disorder comprises at least one selected from the group consisting of acute motor axonal neuropathy (AMAN), Addison's disease, anti-NMDA receptor encephalitis, antiphospholipid syndrome, autoimmune gastritis, autoimmune hepatitis, autoimmune polyendocrine syndrome type 1 (APS-1), celiac disease, choreathetosis, chorea, chronic autoimmune hepatitis, chronic thyroiditis and other auto-immune thyroid diseases, Churg-Strauss syndrome, CREST syndrome, dermatitis herpetiformis, diabetes mellitus type 1 (Type 1 diabetes or T1D), encephalomyelitis, granulomatosis with polyangiitis, Graves' disease, Hashimoto's thyroiditis, inflammatory myopathy, Isaac's Syndrome (autoimmune neuromyotonia), Lambert-Eaton myasthenic syndrome, limbic encephalitis, microscopic polyangiitis, Miller-Fisher Syndrome, Mixed Connective Tissue Disease, multifocal motor neuropathy with conduction block (MMN), multiple sclerosis, myasthenia gravis, neonatal heart block, neuromyelitis optica (Devic's syndrome), opsoclonus myoclonus syndrome, optic neuropathy, paediatric autoimmune neuropsychiatric disease associated with Streptococcus (PANDAS), paraneoplastic cerebellar degeneration, paraneoplastic cerebellar syndrome, polymyositis/dermatomyositis, primary biliary cirrhosis, primary Sjögren's syndrome, rheumatoid arthritis, scleromyositis, scleroderma, Stiff person syndrome, subacute sensory neuronopathy, Sydenham's chorea, systemic lupus erythematosus, systemic sclerosis, and systemic vasculitides.

In certain embodiments, the disease or disorder comprises at least one selected from the group consisting of type 1 diabetes, celiac disease, autoimmune thyroiditis and APS-1. In other embodiments, the disease or disorder comprises type 1 diabetes and/or celiac disease.

In certain embodiments, the kit comprises a plurality of antigens, wherein each of the plurality of antigen specifically, and exclusively from each other, binds to one autoantibody associated with one of the plurality of diseases or disorders, wherein each antigen comprises the antigen derivatized with a detectable label (first derivatized antigen) and the antigen derivatized with a tagging label (second derivatized antigen). In other embodiments, the kit comprises a solid surface comprising a plurality of non-overlapping areas, wherein each one of the plurality of non-overlapping areas is derivatized with a capture molecule that binds with one of the tagging labels, wherein each one of the tagging labels bind to only one of the plurality of non-overlapping areas on the solid surface.

In certain embodiments, the solid substrate surface comprises at least one selected from the group consisting of a silicon wafer, glass, metal, plastic, ceramic, metal alloy, and polymer.

### BRIEF DESCRIPTION OF THE DRAWINGS

The following detailed description of exemplary embodiments of the invention will be better understood when read in conjunction with the appended drawings. For the purpose of illustrating the invention, there are shown in the drawings exemplary embodiments. It should be understood, however, that the invention is not limited to the precise arrangements, number of arrangements combined, and instrumentalities of the embodiments shown in the drawings.

FIG. 1 is a non-limiting illustration of an exemplary multiplex assay. The autoantibodies in serum link each Sulfo-tagged antigen to its corresponding linker-labeled antigen, respectively, which is then captured on the solid phase of the QuickPlex 4-Spot plate. In certain embodiments, detection of plate-captured Sulfo-tagged antigen is accomplished with electrochemiluminescence (ECL).

FIGS. 2A-2B are a series of graphs illustrating levels of 4 autoantibodies, as determined using multiplex ECL assay and radioassay, among 40 newly diagnosed patients with T1D: (FIG. 2A) IAA, (FIG. 2A) IA-2A, (FIG. 2B) GADA, and (FIG. 2B) TGA. The two assays were correlated (P<0.0001), but ECL-IAA and ECL-TGA assays detected more positives than radioassay in patients with 100% specificity in 50 healthy controls.

FIGS. 3A-3B are a series of graphs illustrating levels of 4 autoantibodies, as determined using multiplex ECL assay and single ECL assay, among 40 newly diagnosed patients with T1D: (FIG. 3A) IAA, (FIG. 3A) GADA, (FIG. 3B) IA-2A, and (FIG. 3B) TGA. The two assays had an excellent correlation (p<0.0001), while two low IAA and one low IA-2A in multiplex ECL assay negative in single ECL assay were likely false positives caused by signal interference of very high signal from another antibody.

FIGS. 4A-4C illustrate working charts of the multiplex ECL assay on a non-limiting U-Plex Plate. The autoantibodies in serum link each Sulfo-tagged antigen to its corresponding biotin-labeled antigen, which is then bound by its corresponding linker-labeled Streptavidin, respectively. Each complex is then captured on the solid phase of the multiple-spot UPlex plate and detection of plate-captured Sulfo-tagged antigens is accomplished with electrochemiluminescence. The multiplex ECL assay on UPlex plate was validated in new-onset T1D patients (n=168) and healthy controls (n=118). The positive cut-offs for all autoantibodies were set at the 100^{th} percentile of the 118 controls. The multiplex ECL assay retained 100% sensitivity for all autoantibodies.

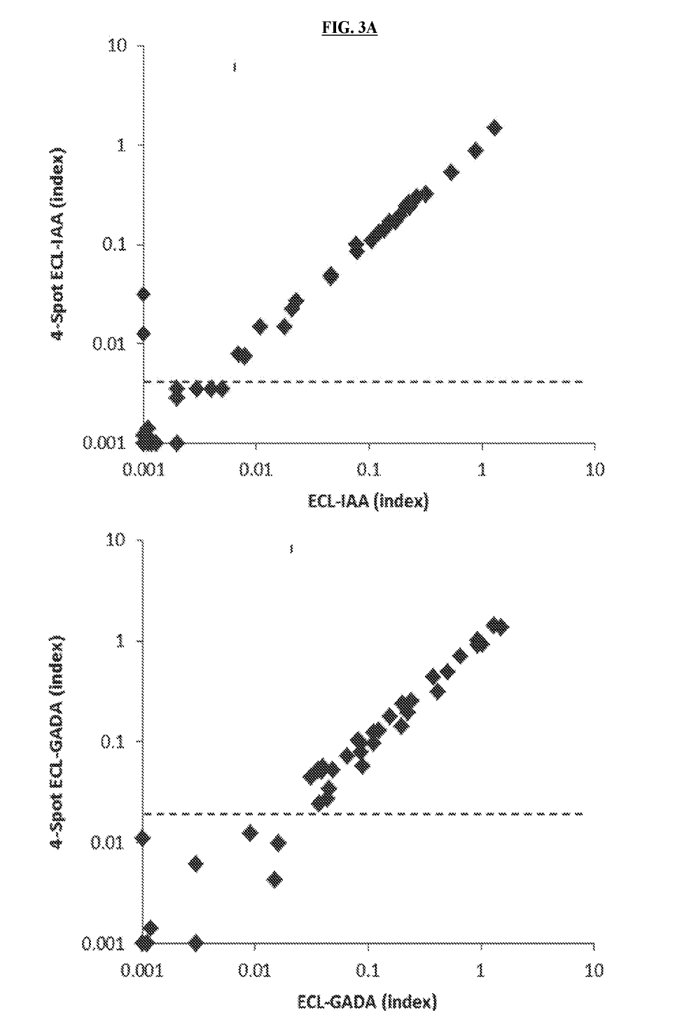

FIG. 5A comprises a graph illustrating correlation for measurement of GADA levels on 168 new onset T1D patients using a non-limiting multiplex (U-Plex Plate) assay with GADA radioassay. The two assays were correlated (P<0.0001).

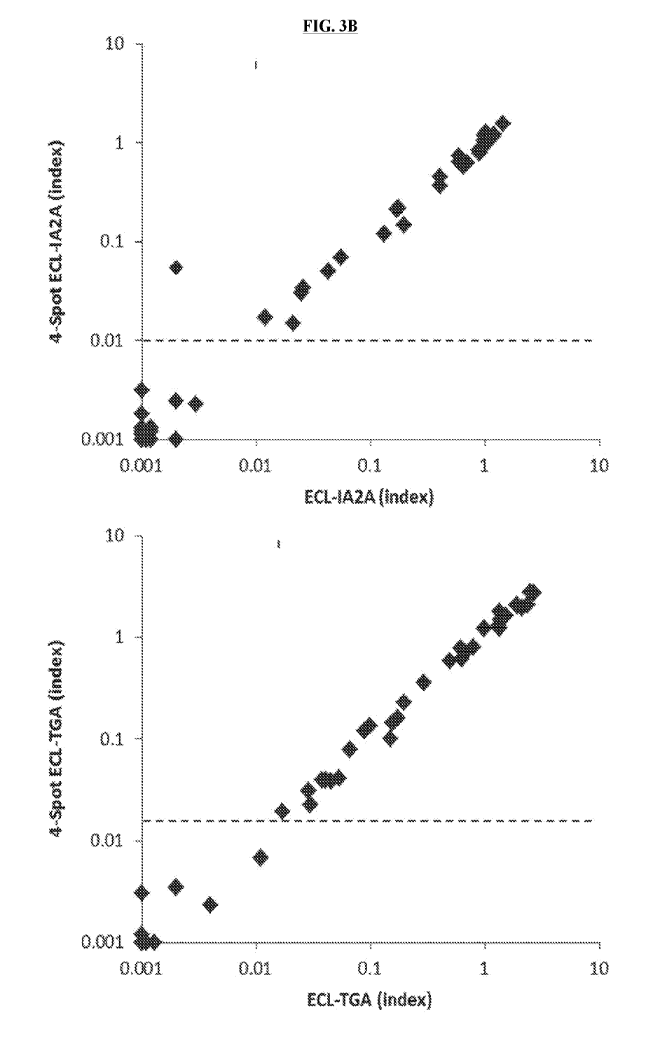

FIG. 5B comprises a graph illustrating correlation for measurement of IA-2A levels on new 168 onset T1D patients using a non-limiting multiplex (U-Plex Plate) assay with IA-2A radioassay. The two assays were correlated (P<0.0001).

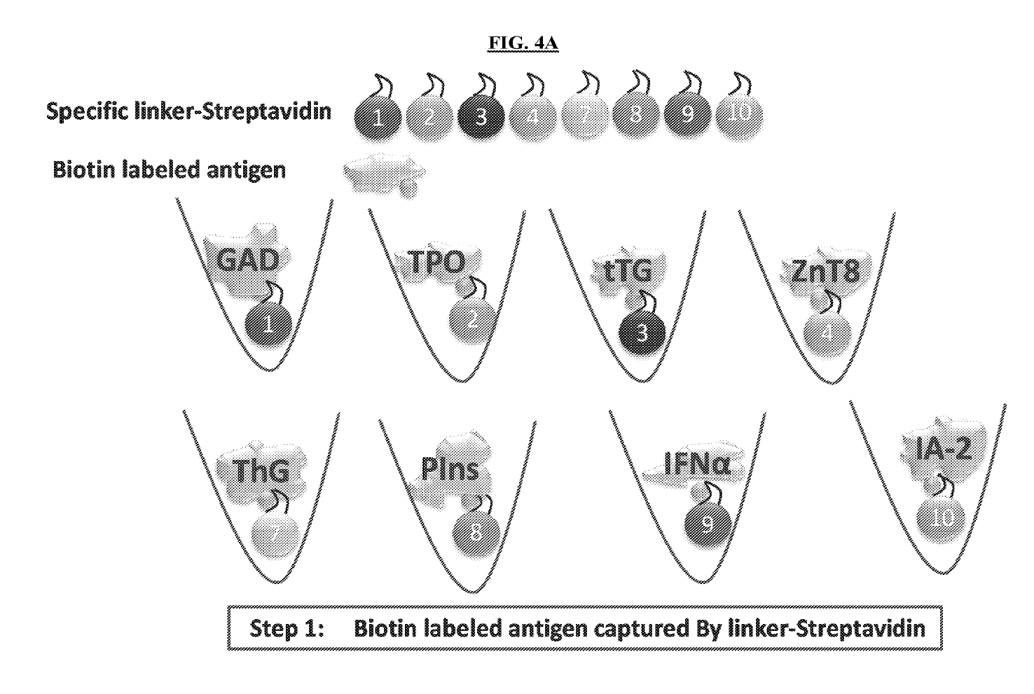

FIG. 5C comprises a graph illustrating correlation for measurement of IAA levels on 168 new onset T1D patients using a non-limiting multiplex (U-Plex Plate) assay with IAA radioassay. The two assays were correlated (P<0.0001).

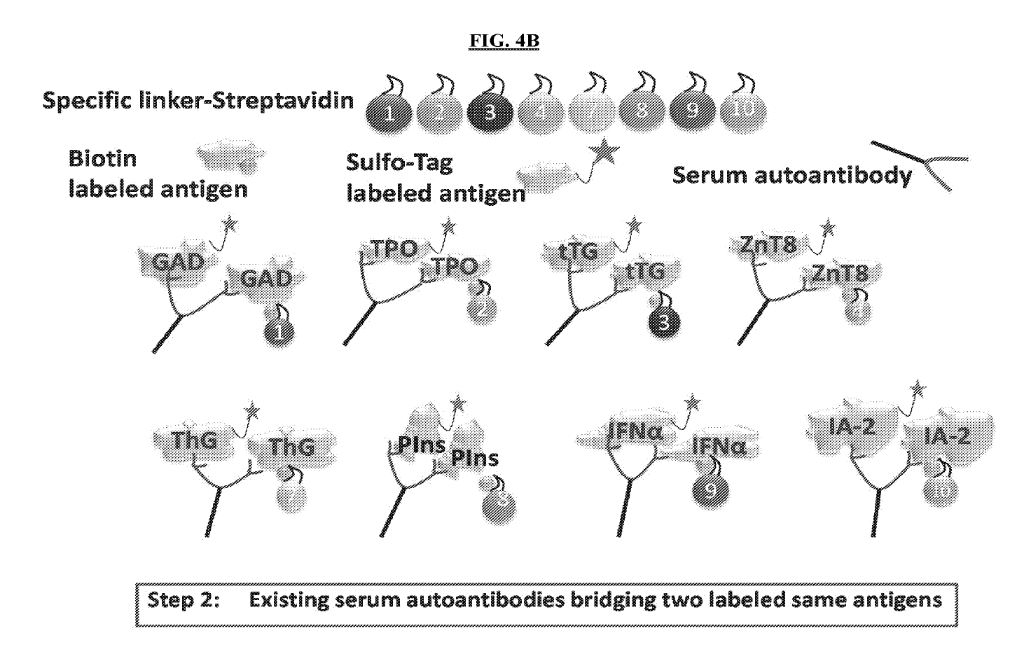

FIG. 5D comprises a graph illustrating correlation for measurement of TGA levels on 168 new onset T1D patients using a non-limiting multiplex (U-Plex Plate) assay with TGA radioassay. The two assays were correlated (P<0.0001).

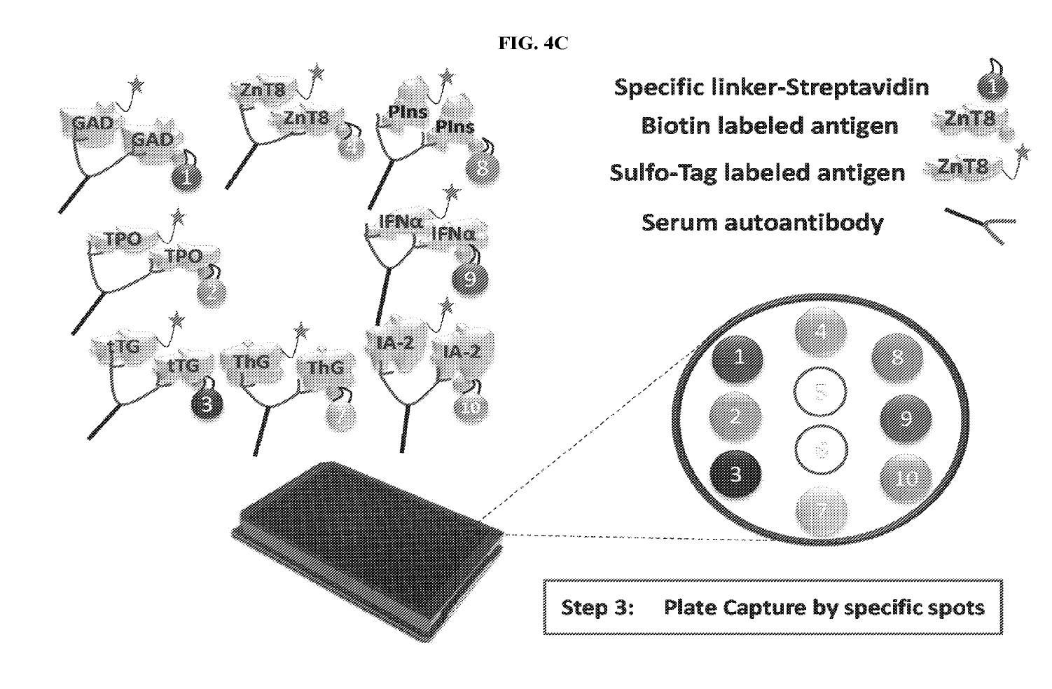

FIG. 5E comprises a graph illustrating correlation for measurement of autoantibodies to thyroid peroxidase (TPOA) levels on 168 new onset T1D patients using a non-limiting multiplex (U-Plex Plate) assay with TPOA radioassay. The two assays were correlated (P<0.0001).

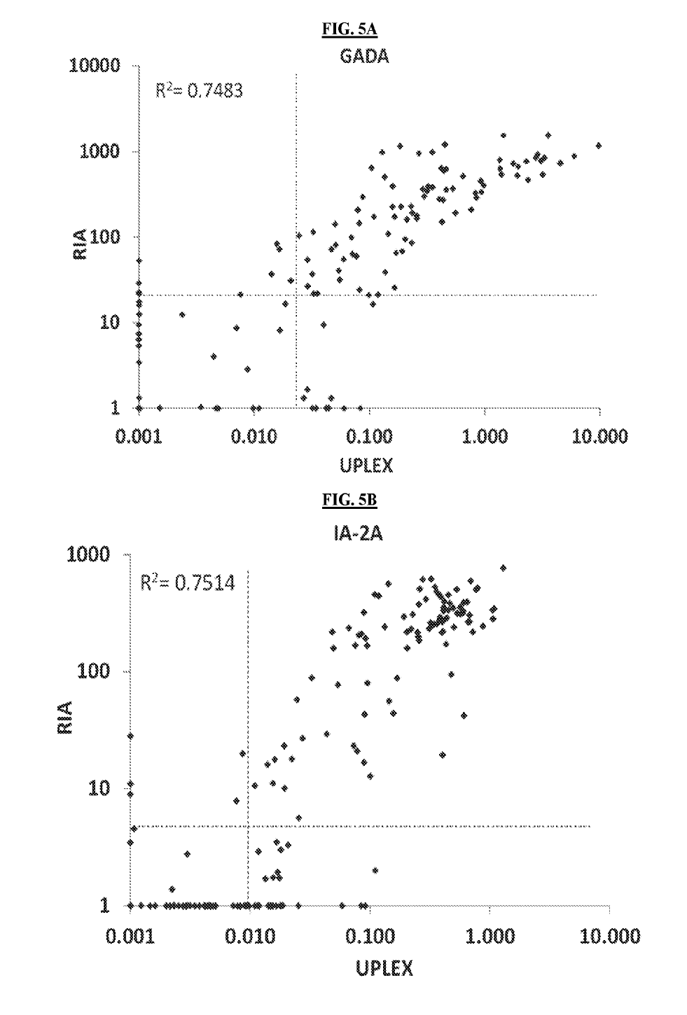

FIG. 5F comprises a graph illustrating correlation for measurement of autoantibodies to thyroid globulin (ThgA) levels on new onset T1D patients using a non-limiting multiplex (U-Plex Plate) assay with ThgA radioassay. The two assays were correlated (P<0.0001).

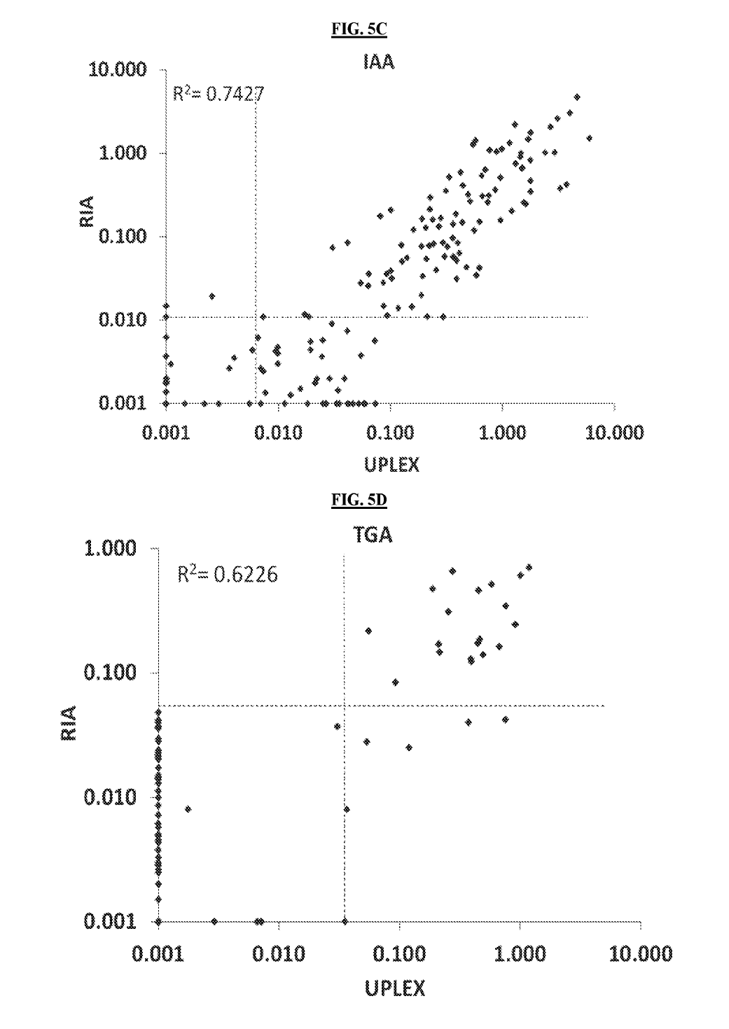

FIG. 5G comprises a graph illustrating correlation for measurement of autoantibodies to interferon alpha (IFNαA) levels on new onset T1D patients using a non-limiting multiplex (U-Plex Plate) assay with IFNαA ELISA. The two assays were correlated (P<0.0001).

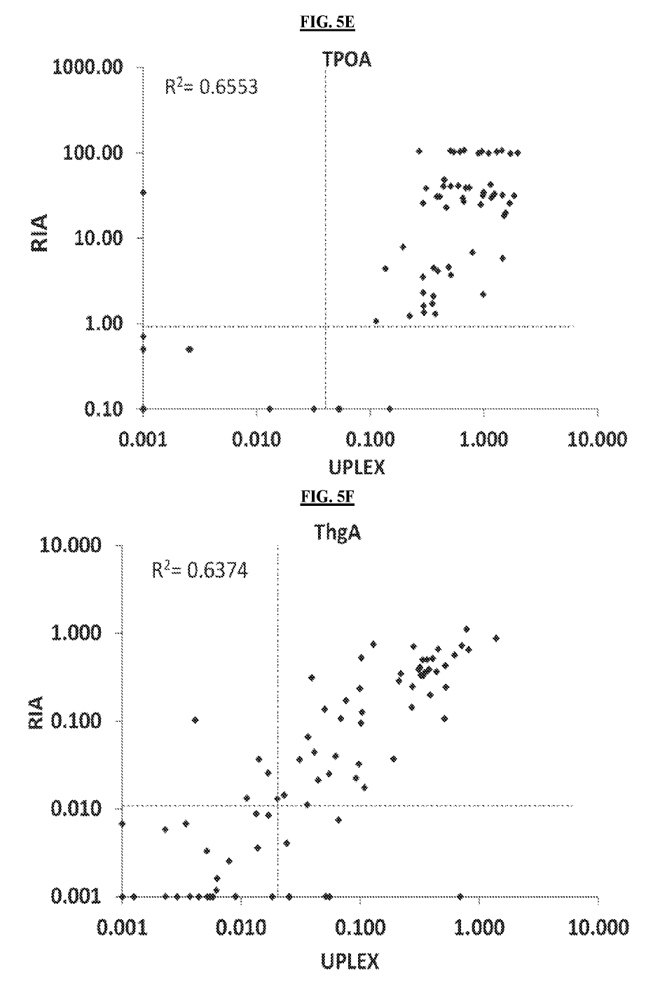

FIG. 5H comprises a graph illustrating correlation for measurement of ZnT8A levels on 48 sample using ECL single assay with ZnT8A radioassay. The two assays were correlated (P<0.0001). The ZnT8A ECL assay can be added on to multiplex ECL assay.

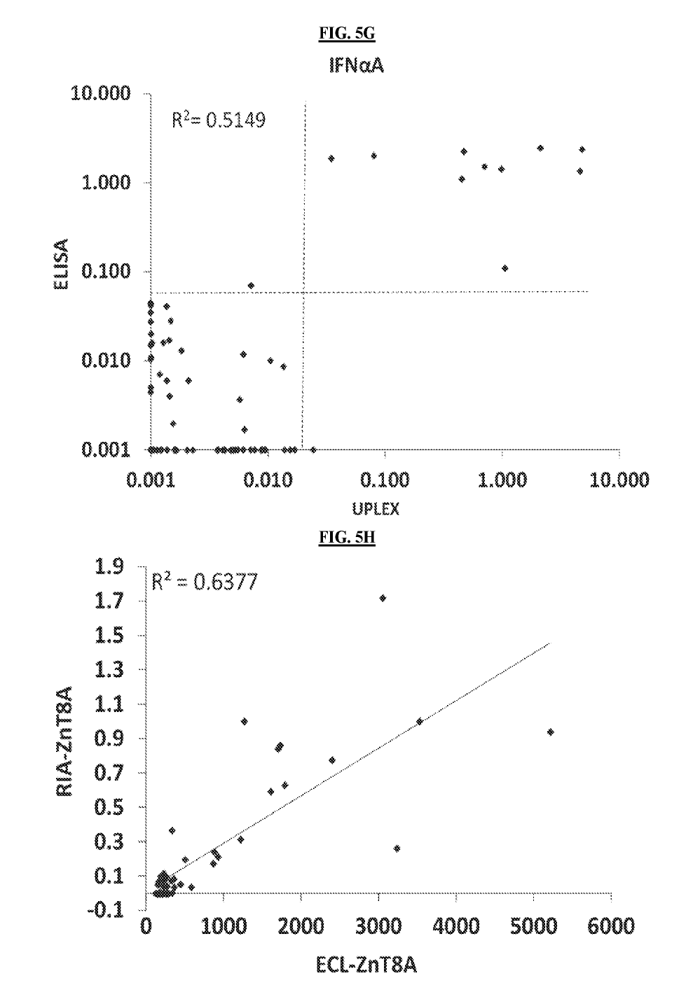

### DETAILED DESCRIPTION OF THE INVENTION

The invention includes methods and kits that can be used to determine simultaneously whether any one antibody out of a plurality thereof is present in a sample. In certain embodiments, at least one of the antibodies comprises an autoantibody. In other embodiments, all of the antibodies are autoantibodies.

In certain embodiments, the novel multiplex autoantibody assay reported herein represents a major progress towards simplification of a large-scale population screening for T1D and/or other autoimmune diseases. In certain embodiments, the ECL-based multiplex assay has a number of attractive features, compared with a combination of the current “gold standard” single autoantibody assays used for TrialNet, The Environmental Determinants of Diabetes in the Young (TEDDY), Immune Tolerance Network (ITN), Type 1 Diabetes Genetic Consortium (T1DGC), the Diabetes Autoimmunity Study in the Young (DAISY) and other studies.

The invention should not be construed to be limited to ECL detection, which is exemplified in a non-limiting manner herein. Molecules (such as antigens and/or antibodies) can be labelled with any known or applicable detectable label, such as but not limited to a SULFO-TAG®, any applicable enzyme (such as, but not limited to, luciferase, sulfatase, phosphatase, and/or peroxidase), a fluorogenic compound, a nucleotide sequence, or the like, as described herein or as known to those skilled in the art.

In one aspect, using a QuickPlex 4-Spot plate with separate linkers for each labeled antigen, four different autoantibody assays can be accommodated in a single well with a small amount of serum sample (6 μl). In certain embodiments, the sensitivity and specificity of the multiplex ECL assay are comparable to the gold standard radioassay measurements in terms of positivity in patients versus normal controls. In other embodiments, more positives are identified in patients regarding IAA. In certain embodiments, the biological sample required by the assay is less than or equal to about 50 μl, less than or equal to about 40 μl, less than or equal to about 30 μl, less than or equal to about 20 μl, less than or equal to about 10 μl, less than or equal to about 9 μl, less than or equal to about 8 μl, less than or equal to about 7 μl, less than or equal to about 6 μl, or less than or equal to about 5 μl. In other embodiments, the biological sample required by the assay is μl, equal to about 50 μl, equal to about 40 μl, equal to about 30 μl, equal to about 20 μl, equal to about 10 μl, equal to about 9 μl, equal to about 8 μl, equal to about 7 μl, equal to about 6 μl, or equal to about 5 μl.

The single ECL assays for IAA and GADA are extensively validated in samples obtained from DAISY, TrialNet Pathway to Prevention, and TEDDY study. ECL-IAA is superior to the current standard mIAA radioassay for its higher sensitivity and earlier identification of iAb seroconversion among young children. Without wishing to be limited by any theory, tin certain embodiments this is due to the ECL assays' ability to detect autoantibodies in all immunoglobulin classes, including IgM. The single ECL-TGA assay is also more sensitive than the standard TGA (IgA) radioassay and can identify TGA seroconversion earlier than the TGA radioassay among DAISY young children who converted to TGA positivity and were confirmed with clinical celiac disease by biopsy. Both ECL-IAA and ECL-GADA assays discriminate high-affinity, high-risk autoantibodies from those “low risk”, low-affinity signals in subjects who have not progressed to T1D. Nevertheless, the detection methods contemplated within the invention are not limited to ECL. In fact, any detection method that provides sufficient spatial resolution for a plurality of antibodies to be probed simultaneously using minimal amount of biological sample can be used within the methods of the invention, as described elsewhere herein.

The assay conditions in the present studies are analogous to those in single ECL assay protocols. The background and signals of one autoantibody did not interfere with autoantibody measurements of neighboring spots in a certain range. Three false positive results (out of 160 measurements), compared to single ECL measurements, were observed. Two false positive IAA results were likely caused by interference of very high signals (both above 25,000 counts) from neighboring spots. One false positive IA-2A level in the combined assay was also likely due to an extremely high signal (>40,000 counts) from its neighboring spot. In general, signals of less than 20,000 counts did not interfere with each other. In certain embodiments, optimization of reaction conditions may encompass adjusting the ratio and amounts of two or more differently labeled antigen proteins. Alternatively, samples with a very high signal maybe re-tested using single autoantibody assays if optimization is not ideal. Such testing may be required in only about 0.2% of samples positive for an iAb in light of general population screening.

As expected, the multiplex ECL assay did not lose iAb positivity among the new onset T1D patients compared to the radioassay results. Furthermore the multiplex assay maintained perfect specificity in 50 healthy controls for all 4 autoantibodies.

An ELISA-based ElisaRSR™ 3 Screen ICA™ is available from the RSR Limited (Cardiff, the U.K.). This combination assay detects GADA, IA-2A and ZnT8A, in separate wells. Non-limiting advantages of the present ECL multiplex assay compared with the 3 Screen ICA™ assay include: ability to detect IAA—the primary iAb in children; ability to screen for two diseases—pre-T1D and celiac disease; and much smaller required serum volume, for duplicate measurements—12 μl vs. 150 μl. Further, the present assay is radioactivity-free and high-throughput.

In certain embodiments, the assay detects the presence of ZnT8A in the mammal's biological sample. In other embodiments, the assay does not detect the presence of ZnT8A in the mammal's biological sample. ZnT8A alone is present in only 1% of subjects followed to clinical diabetes, and in two large studies (TrialNet and TEDDY) ZnT8A was not used for initial screening and the ZnT8A assay was performed only if another iAb is positive. In certain embodiments, TGA is more prevalent in T1D patients than iAbs. In other embodiments, combined screening for iAbs and TGA is more therapeutically valuable and attractive than screening for iAbs alone.

By using ECL detection on a platform from MesoScale Discovery, a multiplex assay was developed to accurately measure all four autoantibodies in a single well using a small blood volume. Such assay facilitates large-scale, general population screening simultaneously for T1D and celiac disease risk.

In another aspect, as demonstrated herein, using ECL detection on a platform of UPlex from MesoScale Discovery, it was possible to perform multiplex measurement, up to 10 antibodies in a single well. As demonstrated herein, the present studies successfully combined IAA, GADA, IA-2A, TPOA, ThgA, TGA, IFNαA and ZnT8A to develop a 8-plex assay, which allows for simultaneous screening of T1D and celiac disease, autoimmune thyroiditis and APS-1 that frequently seen in children with T1D.

The invention further contemplates using additional autoantibodies that allow for detection of autoimmune diseases. Non-limiting examples of such autoantibodies include 21-hydroxylase autoantibodies for Addison's disease; Cyclic Citrullinated Peptide (CCP) autoantibodies for rheumatoid arthritis (RA); anti-double stranded DNA (dsDNA) autoantibodies and antinuclear antibodies (ANA) for SLE (Systemic lupus erythematosus; also known as Lupus), scleroderma, Sjögren's syndrome, polymyositis/dermatomyositis, mixed connective tissue disease, and autoimmune hepatitis; ATPase autoantibodies for autoimmune gastritis; and/or anti-myelin basic protein (MBP) autoantibodies for multiple sclerosis (MS).

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

As used herein, the articles “a” and “an” are used to refer to one or to more than one (i.e., to at least one) of the grammatical object of the article. By way of example, “an element” means one element or more than one element.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type, might be abnormal for a different cell or tissue type.

As used herein, “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “ANA” refers to antinuclear antibody.

The term “antibody,” as used herein, refers to an immunoglobulin molecule that is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, synthetic antibodies, chimeric antibodies, and a humanized antibodies (Harlow, et al., 1999, Using Antibodies: A Laboratory Manual, Cold Spring Harbor Laboratory Press, NY; Harlow, et al., 1989, Antibodies: A Laboratory Manual, Cold Spring Harbor, N.Y.; Houston, et al., 1988, Proc. Natl. Acad. Sci. USA 85:5879-5883; Bird, et al., 1988, Science 242:423-426).

As used herein, the term “CCP” refers to Cyclic Citrullinated Peptide.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, nasal, pulmonary and topical administration.

By “detectable label” is meant a composition that when linked to a molecule of interest renders the latter detectable, via spectroscopic, photochemical, biochemical, immunochemical, or chemical means. For example, detectable labels include radioactive isotopes, magnetic beads, metallic beads, colloidal particles, fluorescent dyes, electron-dense reagents, enzymes (for example, as commonly used in an ELISA), biotin, digoxigenin, or haptens. The labeling of an antigen can be carried out by any generally known method. Examples of the detectable label known to those skilled in the art include a fluorescent dye, an enzyme, a coenzyme, a chemiluminescent substance or a radioactive substance. Specific examples may include radioisotopes (³²P, ¹⁴C, ¹²⁵I, ³H, ¹³¹I and the like), fluorescein, rhodamine, dansyl chloride, umbelliferone, luciferase, peroxidase, alkaline phosphatase, beta-galactosidase, beta-glucosidase, horseradish peroxidase, glucoamylase, lysozyme, saccharide oxidase, microperoxidase, biotin and the like.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “dsDNA” refers to double-stranded DNA.

As used herein, the term “ECL” refers to electrochemiluminescence.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

As used herein, the term “GADA” refers to a glutamic acid decarboxylase autoantibody.

As used herein, the term “iAb” refers to an islet autoantibody.

As used herein, the term “IA-2A” refers to an islet antigen 2 autoantibody

As used herein, the term “IAA” refers to an insulin autoantibody.

As used herein, the term “IFNαA” refers to an interferon alpha autoantibody.

As used herein, an “immunoassay” refers to any binding assay that uses an antibody capable of binding specifically to a target molecule to detect and quantify the target molecule.

The term “immunoglobulin” or “Ig” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

“Instructional material” as that term is used herein includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

As used herein, the term “MBP” refers to anti-myelin basic protein.

As used herein, the term “MS” refers to multiple sclerosis.

The terms “patient,” “subject” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

The term “prevent,” “preventing” or “prevention,” as used herein, means avoiding or delaying the onset of symptoms associated with a disease or condition in a subject that has not developed such symptoms at the time the administering of an agent or compound commences.

As used herein, the term “RA” refers to rheumatoid arthritis.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

As used herein, the term “SLE” refers to systemic lupus erythematosus, or lupus.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, “substantially purified” refers to being essentially free of other components. For example, a substantially purified polypeptide is a polypeptide which has been separated from other components with which it is normally associated in its naturally occurring state.

As used herein, the term “T1D” refers to type 1 diabetes.

As used herein, the term “TGA” refers to a transglutaminase autoantibody.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

As used herein, the term “ThgA” refers to a thyroglobulin autoantibody.

As used herein, the term “TPOA” refers to a thyroperoxidase autoantibody.

As used herein, the term “ZnT8A” refers to a zinc transporter type 8 anutoantibody.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.1, 5.3, 5.5, and 6. This applies regardless of the breadth of the range.

### Methods

The invention provides a method of simultaneously detecting the presence or absence of each one of a plurality of antibodies in a sample.

In certain embodiments, the method comprises (1) contacting the sample with an antigen for each one of the plurality of antibodies, wherein each antigen comprises the antigen derivatized with a detectable label (first derivatized antigen) and the antigen derivatized with a tagging label (second derivatized antigen), thus forming a second solution, wherein a first derivatized antigen-antibody-second derivatized antigen complexes is formed if the corresponding antibody is present in the sample. In other embodiments, the method comprises (2) detecting the presence or absence of each one of the first derivatized antigen-antibody-second derivatized antigen complexes using detection of the detectable label.

In certain embodiments, at least one of the plurality of antibodies comprise an autoantibody. In other embodiments, each one of the plurality of antibodies comprises an autoantibody.

In certain embodiments, the sample comprises a biological sample from a mammal. In other embodiments, the biological sample comprises urine, blood, serum, plasma and/or saliva from the mammal.

In certain embodiments, step (2) comprises contacting the second solution with a solid surface comprising a plurality of non-overlapping areas, wherein each one of the plurality of non-overlapping areas is derivatized with a capture molecule that binds specifically to one of the tagging labels, wherein each one of the tagging labels does not bind with more than one of the plurality of non-overlapping areas, thereby immobilizing each of the first derivatized antigen-antibody-second derivatized antigen complexes, if present in the second solution, in a different non-overlapping area of the solid surface.

In certain embodiments, step (1) comprises, independently for each one of the plurality of antibodies, one of the following steps: (a) contacting the sample with an antigen for one of the plurality of antibodies, wherein the antigen comprises the antigen derivatized with a detectable label, thus forming a first solution; and subsequently contacting the first solution with an antigen for the same antibody, wherein the antigen comprises the antigen derivatized with a tagging label; (b) contacting the sample with an antigen for one of the plurality of antibodies, wherein the antigen comprises the antigen derivatized with a tagging label, thus forming a first solution; and subsequently contacting the first solution with an antigen for the same antibody, wherein the antigen comprises the antigen derivatized with a detectable label; and (c) contacting the sample with an antigen for one of the plurality of antibodies, wherein the antigen comprises the antigen derivatized with a tagging label and the antigen derivatized with the detectable label.

In certain embodiments, the detectable label comprises an electrochemiluminescence (ECL) label, enzyme [such as, but not limited to, luciferase, sulfatase, phosphatase (e.g., alkaline phosphatase), beta-galactosidase, glucoamylase, beta-glucosidase, lysozyme, saccharide oxidase, microperoxidase, and/or peroxidase (e.g., horseradish peroxidase)], a fluorogen, and/or a nucleotide sequence. In other embodiments, the detectable label comprises a radioactive isotope (such as, but not limited to, ³²P, ¹⁴C, ¹²⁵I, ³H, ¹³¹I and the like), magnetic bead, metallic bead, colloidal particle, fluorescent dye, electron-dense reagent, chemiluminescent dye, enzyme, co-enzyme, biotin, digoxigenin, and/or hapten. Non-limiting examples of dyes include fluorescein, rhodamine, dansyl chloride, and umbelliferone.

In certain embodiments, the detectable label comprises an ECL label. In other embodiments, the ECL label comprises a ruthenium complex. In other embodiments, the ECL label comprises [Ru(BPy)₃]²⁺, where BPy is 2,2′-bipyridine.

In certain embodiments, the tagging label comprises biotin, carbohydrate, immunoglobulin or fragment thereof, or any combinations thereof. In other embodiments, the capture molecule comprises avidin, streptavidin, lectin, protein A/G, an aptamer, or any combinations thereof.

In certain embodiments, the plurality of antibodies comprises at least four antibodies. In other embodiments, the plurality of antibodies comprises at least six antibodies. In yet other embodiments, the plurality of antibodies comprises at least eight antibodies. In yet other embodiments, the plurality of antibodies comprises at least ten antibodies.

In certain embodiments, the plurality of antibodies comprises at least one selected from the group consisting of IAA, GADA, IA-2A, TPOA, ThgA, TGA, IFNαA and ZnT8A.

In certain embodiments, the plurality of antibodies comprises at least one selected from the group consisting of IAA, GADA, IA-2A, TPOA, ThgA, TGA, IFNαA, ZnT8A, 21-hydroxylase autoantibody, CCP autoantibody, anti-dsDNA autoantibody, ANA, ATPase autoantibody, and anti MBP autoantibody.

In certain embodiments, the plurality of antibodies comprises IAA, GADA, IA-2A, TPOA, ThgA, TGA, IFNαA and/or ZnT8A. In certain embodiments, the plurality of antibodies comprises IAA, GADA, IA-2A, TPOA, ThgA, TGA, IFNαA and ZnT8A. In yet other embodiments, the plurality of antibodies comprises IAA, GADA, IA-2A, and TGA.

In certain embodiments, the mammal is human.

The invention further provides a method of determining if a mammal is likely to develop or has developed each one of a plurality of diseases or disorders, wherein each one of the plurality of diseases or disorders is characterized by the presence of at least one autoantibody in a biological sample of the mammal. In certain embodiments, the method comprises: (1) contacting the sample with a plurality of antigens, wherein each of the plurality of antigens specifically, and exclusively from each other, binds to one autoantibody associated with one of the plurality of diseases or disorders, wherein each antigen comprises the antigen derivatized with a detectable label (first derivatized antigen) and the antigen derivatized with a tagging label (second derivatized antigen), thus forming a second solution; and (2) detecting the presence or absence of each first derivatized antigen-autoantibody-second derivatized antigen complexes using ECL detection

In certain embodiments, the presence of a first derivatized antigen-autoantibody-second derivatized antigen indicates that the mammal is likely to develop or has developed the disease or disorder associated with the autoantibody.

In certain embodiments, the disease or disorder comprises an autoimmune disease. In other embodiments, the disease or disorder comprises at least one selected from the group consisting of acute motor axonal neuropathy (AMAN), Addison's disease, anti-NMDA receptor encephalitis, antiphospholipid syndrome, autoimmune gastritis, autoimmune hepatitis, autoimmune polyendocrine syndrome type 1 (APS-1), celiac disease, choreathetosis, chorea, chronic autoimmune hepatitis, chronic thyroiditis and other auto-immune thyroid diseases, Churg-Strauss syndrome, CREST syndrome, dermatitis herpetiformis, diabetes mellitus type 1 (Type 1 diabetes or T1D), encephalomyelitis, granulomatosis with polyangiitis, Graves' disease, Hashimoto's thyroiditis, inflammatory myopathy, Isaac's Syndrome (autoimmune neuromyotonia), Lambert-Eaton myasthenic syndrome, limbic encephalitis, microscopic polyangiitis, Miller-Fisher Syndrome, Mixed Connective Tissue Disease, multifocal motor neuropathy with conduction block (MMN), multiple sclerosis, myasthenia gravis, neonatal heart block, neuromyelitis optica (Devic's syndrome), opsoclonus myoclonus syndrome, optic neuropathy, paediatric autoimmune neuropsychiatric disease associated with Streptococcus (PANDAS), paraneoplastic cerebellar degeneration, paraneoplastic cerebellar syndrome, polymyositis/dermatomyositis, primary biliary cirrhosis, primary Sjögren's syndrome, rheumatoid arthritis, scleromyositis, scleroderma, Stiff person syndrome, subacute sensory neuronopathy, Sydenham's chorea, systemic lupus erythematosus, systemic sclerosis, and systemic vasculitides. In yet other embodiments, the disease or disorder comprises T1D or celiac disease. In yet other embodiments, the disease or disorder comprises T1D and celiac disease. In yet other embodiments, the disease or disorder comprises T1D, celiac disease, autoimmune thyroiditis and APS-1.

In certain embodiments, if the mammal is likely to develop or has developed one of the diseases or disorders, the mammal is administered medication and/or therapy that prevents or treats the disease or disorder.

In certain embodiments, if the mammal is likely to develop or has developed one of the diseases or disorders, the mammal is counseled to seek medication and/or therapy that prevents or treats the disease or disorder.

The invention further provides a kit for simultaneously determining if a mammal is likely to develop or has developed each one of a plurality of diseases or disorders, wherein each one of the plurality of diseases or disorders is characterized by the presence of at least one autoantibody in a biological sample of the mammal. In certain embodiments, the kit comprises a plurality of antigens, wherein each of the plurality of antigen specifically, and exclusively from each other, binds to one autoantibody associated with one of the plurality of diseases or disorders, wherein each antigen comprises the antigen derivatized with a detectable label (first derivatized antigen) and the antigen derivatized with a tagging label (second derivatized antigen). In certain embodiments, the kit comprises a solid surface comprising a plurality of non-overlapping areas, wherein each one of the plurality of non-overlapping areas is derivatized with a capture molecule that binds with one of the tagging labels, wherein each one of the tagging labels bind to only one of the plurality of non-overlapping areas on the solid surface.

In certain embodiments, the solid substrate surface comprises a silicon wafer, glass, metal, plastic, ceramic, metal alloy, polymer or any combinations thereof.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Materials and Methods

### Subjects:

Serum samples were obtained from 40 newly diagnosed T1D patients and 50 healthy controls. All T1D patients were positive for at least one iAb by a standard radioassay and 19/40 were TGA positive by radioassay. The healthy controls were of similar age as the cases and negative for all iAbs and TGA by radioassay.

### Multiple Autoantibody Multiplex ECL Assay:

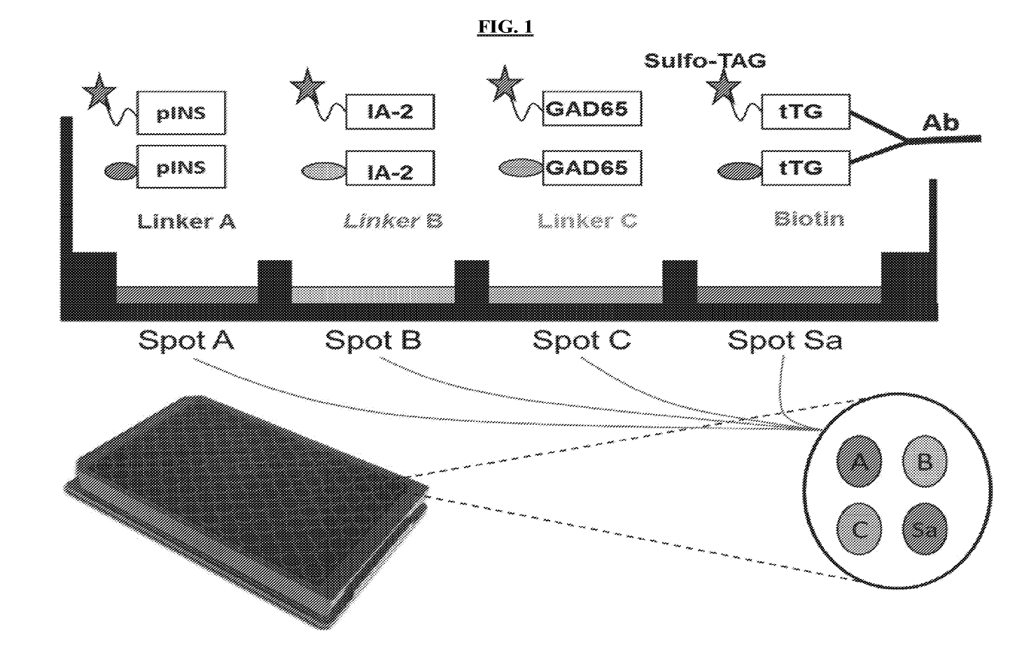

Previously described individual ECL IAA and GADA assays were adapted to measure four antibodies as illustrated in FIG. 1. Briefly, 12 μl of patient serum (the amount sufficient for duplicate measurement) were mixed with 14.5 μl of 500 mM of acetic acid, which aids in IAA determination. After incubation for 45 minutes at room temperature, 25 μl of the acid treated serum solution were transferred to a 96-well plate with freshly prepared antigen/neutralization solution consisting of 8.3 μl of 1M Tris-HCl (pH=9.0) and 35 μl of labeled antigen mixture (Sulfo-TAG and linker-A labeled proinsulin at concentration of 100 ng/ml; Sulfo-TAG and linker-B labeled IA-2 at the concentration of 125 and 250 ng/ml respectively; Sulfo-TAG and linker-C labeled GAD65 at the concentration of 125 and 500 ng/ml respectively; Sulfo-TAG and biotin labeled transglutaminase at the concentration of 100 and 400 ng/ml respectively) in PBS with 5% BSA (v/v).

The mixture was incubated at room temperature for 2 hours with agitation, followed by incubation at 4° C. overnight (>16 hours). On the same day, QuickPlex 4-Spot plates were blocked with 150 μl of 3% Blocker A (MSD) per well overnight at 4° C. On the second day, the blocked 4-Spot plate was washed with PBST (PBS with 0.05% Tween-20) three times, followed by addition of the overnight incubated serum mixture. The components from each well were divided into two wells, 30 μl per well on a 4-Spot plate. After incubation at room temperature for 1 hour followed by 3 washes with PBST to remove excess labeled antigens, 150 μl/well of 2× Read buffer (MSD) were added and the plate was counted on a MSD Sector Imager 2400 (MSD, Rockville, Md.). A mouse monoclonal insulin antibody-125 (provided by Dr. Tom Thomas, Vanderbilt), a mouse monoclonal GAD65 antibody (GAD-6, Abcam, Cambridge, Mass.), a standard internal IA-2A positive serum, and a standard internal TGA positive serum were used as the assay internal standard positive controls for the 4 autoantibodies, respectively, and the results for all 4 autoantibodies were expressed as an index (index=[Signalₛₐₘₚₗₑ−Signal_{NegativeControl}]/[Signal_{PositiveControl}−Signal_{NegativeControl}]. The assay cut-offs for the 4 autoantibodies in this multiplexed ECL assay were referred to their corresponding single ECL assays.

### Radioassay

The radioassays for mIAA, GADA, IA-2A, and TGA used in the present study were all performed as described in Bao, et al., 1999, J. Autoimmun. 13:143-148; Yu, et al., 2000, Proc Natl Acad Sci. 97:1701-1706; Bonifacio, et al., 2010, J. Clin. Endocrinol. Metab. 95:3360-3367. The radioassay cut-offs were set at the 99^{th} percentile of 500 normal control samples for GADA and IA-2A and 106 controls for mIAA, respectively. The cut-off for the TGA radioassay was set at 100^{th} percentile of 184 normal control samples.

### Statistics

Statistical analyses were performed using correlation analysis, rank sum or Fisher's exact test in PRISM 6.0 software (GraphPad Software Inc., San Diego, Calif.). A p-value<0.05 was considered statistically significant.

### Example 1

This non-limiting example relates to the multiplex detection of autoantibodies IAA, GADA, IA-2A and TGA using electrochemiluminescense (ECL) detection, as a way to simultaneously screen for risk of T1D and celiac disease. In certain embodiments, the present method may be used for mass screening of large population.

Of the 40 newly diagnosed patients with T1D, 19 patients, 31 patients, 24 patients and 19 patients were positive for, respectively, mIAA, GADA, IA-2A, and TGA. The ECL multiplex assay detected IAA, GADA, IA-2A, and TGA in, respectively, 26 patients, 31 patients, 26 patients and 31 patients. Thus the ECL multiplex assay detected mIAA, IA-2, and TGA (but not GADA) in more patients than the radioasssay. The increase of positivity in patients was especially evident for IAA (26 versus 19; p=0.17) and TGA (31 versus 19; p=0.01). All control samples tested negative for all iAbs and TGA in the ECL multiplex assay.

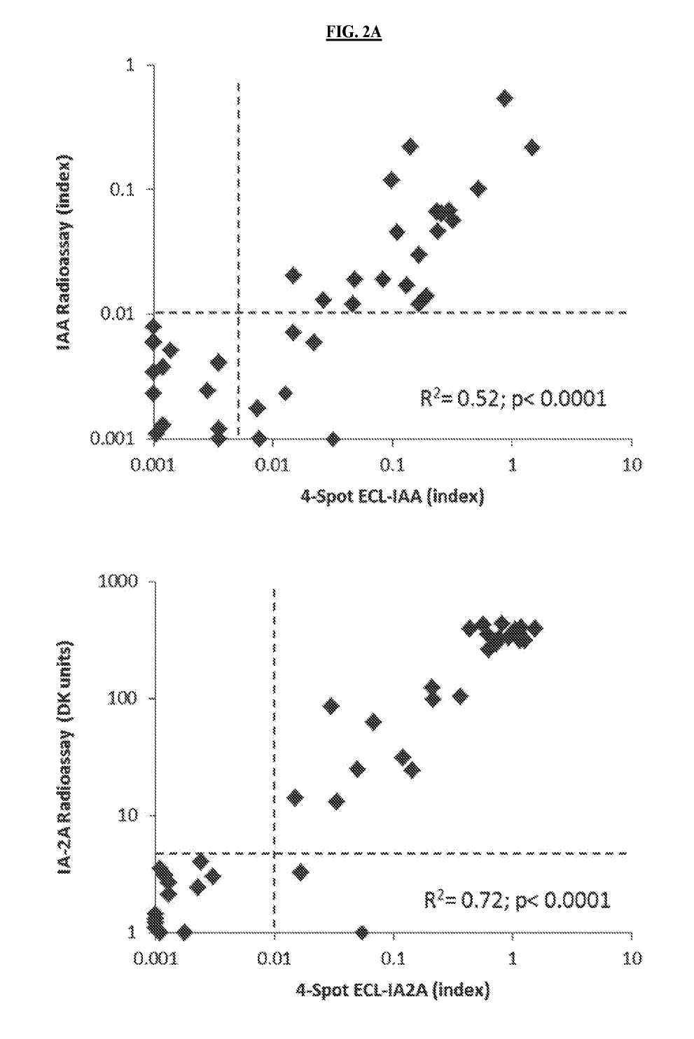

The levels of autoantibodies in the ECL multiplex assay correlated well with the corresponding single-antibody radioassays (FIGS. 2A-2B), even though ECL-IAA and ECL-TGA assays detected more positives.

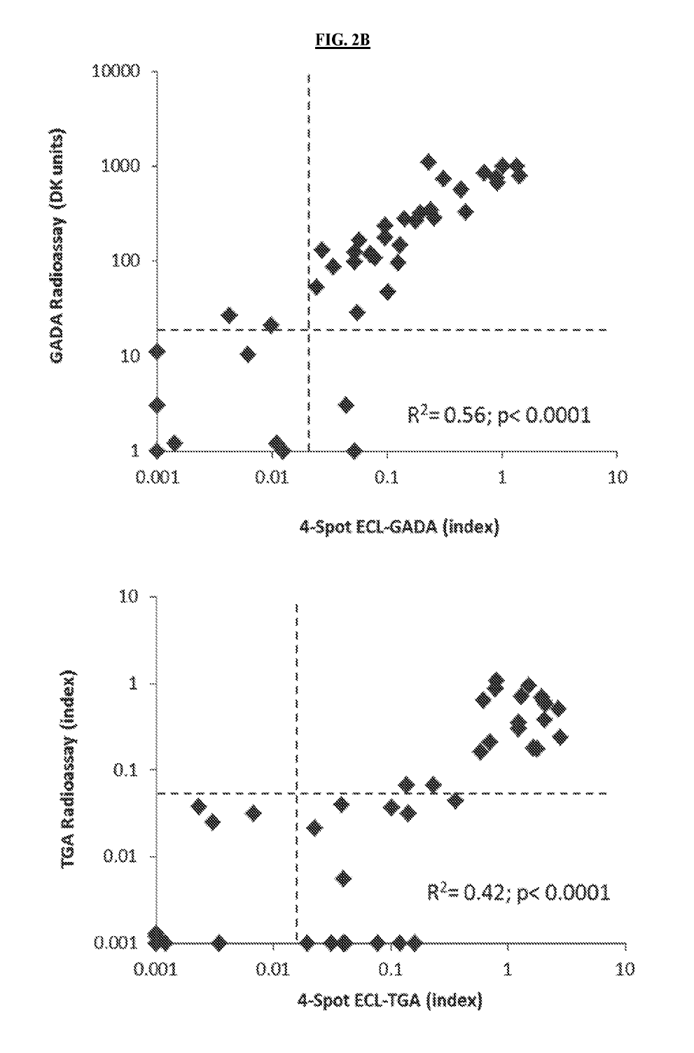

To evaluate a potential signal interference from neighboring spots, each antibody measurement in the multiplex ECL assay was compared to an individual antibody ECL assay (FIGS. 3A-3B). Only 3/160 paired comparisons showed discrepancies that could be attributed to signal interference: two for IAA (Panel A) and one for IA-2A (Panel B). All three positive results in the multiplex ECL assay corresponding to negative single ECL antibody assays were at a low level and were accompanying another high autoantibody signal in the same well. These three low level positives were very likely false-positives in the multiplex ECL assay caused by signal interference.

At the current time, multiple candidate interventions are proposed to abrogate or slow progression to type 1 diabetes (T1D) among islet autoantibody (iAb) positive subjects, but mass screening for eligible subjects and the general population remains a laborious and inefficient process. As demonstrated herein, a validated nonradioactive iAb assays was developed using electrochemiluminescense (ECL) detection with an excellent sensitivity and specificity compared to the gold-standard radioassays. Using ECL detection on a platform from MesoScale Discovery (MSD) allows the measurement of at least four antibodies in a single well using a small blood volume (6 μl). In the present study using a MSD QuickPlex 4-Spot plate, three iAb to insulin (IAA), GAD65 (GADA), and IA-2 (IA-2A) were successfully combined with tissue transglutaminase autoantibodies (TGA) in a single well of a 96 well plate. Forty new onset T1D patients, all positive for at least one iAb and a half of them positive for TGA by radioassay, were tested as well as 50 healthy controls. The multiplex assay retained 100% sensitivity and 100% specificity for all four autoantibodies in terms of positivity identified in patients versus normal controls compared to the corresponding standard radioassays. The multiplex ECL assay was able to identify more positivity than current radioassays for IAA and TGA. The development of this multiplex assay facilitate high-throughput screening for T1D and celiac disease risk in the general population.

### Example 2

A 7-plex electrochemiluminescence (ECL) assay was developed combining in a single well and using only 6 μl of serum, testing for IAA, GADA and IA-2A, autoantibodies to thyroid peroxidase (TPOA), thyroglobulin (ThgA), transglutaminase (TGA), and interferon alpha (IFNαA). TGA and INFαA are, respectively, markers of celiac disease and autoimmune polyglandular syndrome type 1.

Briefly, 12 μl of patient serum (an amount found to be sufficient for duplicate measurement) were mixed with 14.5 μl of 500 mM of acetic acid, which is necessary for IAA determination. After incubation for 45 minutes at room temperature, 8.8 μl of 1M Tris-HCl (pH=9.0) were added for neutralization. Labeled antigen mixtures were prepared parallel at the same time, but differently from that for multiplex ECL assay with QuickPlex plate as below: each biotin labeled antigen with its optimized amount was mixed with its corresponding linker-streptavidin, respectively, incubated at room temperature for 30 minutes; then, stop solution was added, then its corresponding Sulfo-tagged antigen was added with its optimized amount, respectively, all 7 antigen preparations were mixed together with final volume to 2 ml. 35 μl of antigen mixture per well were transferred into serum preparation well.

The mixture was incubated at room temperature for 2 hours with agitation, followed by incubation at 4° C. overnight (>16 hours). On the same day, UPlex plates were blocked with 150 μl of 3% Blocker A (MSD) per well overnight at 4° C. On the second day, the blocked UPlex plate was washed with PBST (PBS with 0.05% Tween-20) three times, followed by addition of the overnight incubated serum mixture. The components from each well were divided into two wells, 30 μl per well on a UPlex plate. After incubation at room temperature for 1 hour followed by 3 washes with PBST to remove excess labeled antigens, 150 μl/well of 2× Read buffer (MSD) were added, and the plate was counted on a MSD Sector Imager 2400 (MSD, Rockville, Md.). A mouse monoclonal insulin antibody-125 (provided by Dr. Tom Thomas, Vanderbilt), a mouse monoclonal GAD65 antibody (GAD-6, Abcam, Cambridge, Mass.), a standard internal IA-2A positive serum, a standard internal TGA positive serum, a standard internal TPOA positive serum, a standard internal ThgA positive serum, and a standard internal INFαA positive serum were used as the assay internal standard positive controls for the 7 autoantibodies, respectively, and the results for all 7 autoantibodies were expressed as an index (index=[Signalsample−SignalNegativeControl]/[SignalPositiveControl−SignalNegativeControl]. The assay cut-offs for the 7 autoantibodies in this multiplexed ECL assay were referred to their corresponding single ECL assays.

The 7-plex assay was validated in new-onset T1D patients with islet Ab+ (n=168) and healthy controls with islet Ab− (n=118). The positive cut-offs for all autoantibodies were set at the 100^{th} percentile of the 118 controls. Results from the 7-plex assay were compared with those from corresponding single-autoantibody ECL and RIA assays and a standard ELISA assay for IFNαA. Among the T1D patients, the 7-plex assay correlated well in levels with each corresponding single-antibody RIA (R²=0.74, 0.75, 0.75, 0.66, 0.64, 0.62, and 0.51 for IAA, GADA, IA-2A, TPOA, ThgA, TGA and IFNα with ELISA, respectively, p<0.0001 for all). The 7-plex assay retained 100% sensitivity for all autoantibodies and the positivity of 7-plex assay and their corresponding RIA or ELISA in 168 T1D patients is summarized in Table 1.

In terms of specificity on 118 healthy controls, each of one those samples were negative for all 7 Abs. Excellent correlation was observed between 7-plex and radioassay/ELISA. The ZnT8A ECL single assay was validated and showed good correlation with ZnT8A RIA and can be added onto the UPlex assay.

The few discordant samples were at low levels, close to the cut-offs. In conclusion, this novel multiplex assay facilitates screening for T1D, celiac disease, autoimmune thyroiditis and APS-1 in large-scale of general population, using as little as 6 μl of serum.

TABLE 1
Number of subjects positive in the 7-plex assay
and their corresponding RIA or ELISA#.

| | IAA | GADA | IA-2A | TGA | TPOA | ThgA | IFNαA^{#} |
|---|---|---|---|---|---|---|---|
| RIA | 101 | 95 | 102 | 19 | 55 | 51 | 11 |
| 7-Plex | 107 | 97 | 110 | 26 | 57 | 55 | 11 |

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of simultaneously detecting the presence or absence of each one of a plurality of antibodies in a sample, the method comprising the steps of:
(1) contacting a volume of the sample with an antigen for each one of the plurality of antibodies, wherein each antigen comprises the antigen derivatized with a detectable label (first derivatized antigen) and the antigen derivatized with a tagging label (second derivatized antigen), thus forming a second solution, wherein, if a given antibody selected from the plurality of antibodies is present in the sample, a corresponding first derivatized antigen-given antibody-second derivatized antigen complex is formed in the second solution;
(2) contacting the second solution with a solid surface comprising a plurality of non-overlapping areas, wherein each one of the plurality of non-overlapping areas is derivatized with a capture molecule that binds specifically to one of the tagging labels, wherein each one of the tagging labels does not bind with more than one of the plurality of non-overlapping areas, thereby immobilizing each of the first derivatized antigen-given antibody-second derivatized antigen complexes, if present in the second solution, in a different non-overlapping area (which is thereby known to be associated with the given antibody); and
(3) detecting the presence or absence of each one of the first derivatized antigen-given antibody-second derivatized antigen complexes by determining presence or absence of the detectable label immobilized to the non-overlapping area associated with the given antibody.

2. The method of claim 1, wherein at least one of the plurality of antibodies comprise an autoantibody.

3. (canceled)

4. The method of claim 1, wherein the sample comprises a biological sample from a mammal.

5. The method of claim 4, wherein the biological sample from the mammal comprises at least one selected from the group consisting of urine, blood, serum, plasma and saliva.

6. The method of claim 1, wherein the detectable label comprises an electrochemiluminescence (ECL) label.

7. The method of claim 6, wherein the ECL label comprises a ruthenium complex.

8. The method of claim 7, wherein the ECL label comprises [Ru(BPy)₃]²⁺.

9. The method of claim 1, wherein step (1) comprises, independently for each one of the plurality of antibodies, one of the following steps:
(a) contacting the sample with an antigen for one of the plurality of antibodies, wherein the antigen comprises the antigen derivatized with a detectable label, thus forming a first solution; and subsequently contacting the first solution with an antigen for the same antibody, wherein the antigen comprises the antigen derivatized with a tagging label;
(b) contacting the sample with an antigen for one of the plurality of antibodies, wherein the antigen comprises the antigen derivatized with a tagging label, thus forming a first solution; and subsequently contacting the first solution with an antigen for the same antibody, wherein the antigen comprises the antigen derivatized with a detectable label; and
(c) contacting the sample with an antigen for one of the plurality of antibodies, wherein the antigen comprises the antigen derivatized with a tagging label and the antigen derivatized with a detectable label.

10. The method of claim 1, wherein the tagging label comprises at least one selected from the group consisting of biotin, carbohydrate, and immunoglobulin or fragment thereof.

11. The method of claim 1, wherein the capture molecule comprises at least one selected from the group consisting of avidin, streptavidin, lectin, protein A/G, and an aptamer.

12. The method of claim 1, wherein the plurality of antibodies comprises at least four, six, eight, or ten antibodies.

13. (canceled)

14. (canceled)

15. (canceled)

16. The method of claim 1, wherein the volume of the sample ranges from about 6 μL to about 20 μL.

17. The method of claim 1, wherein the plurality of antibodies comprises at least one selected from the group consisting of IAA (insulin autoantibody), GADA (glutamic acid decarboxylase autoantibody), IA-2A (islet antigen 2 autoantibody), TGA (transglutaminase autoantibody), TPOA (thyroperoxidase autoantibody), ThgA (thyroglobulin autoantibody), IFNαA (interferon alpha autoantibody), ZnT8A (zinc transporter type 8 autoantibody), 21-hydroxylase autoantibody, Cyclic Citrullinated Peptide (CCP) autoantibody, anti-double stranded DNA (dsDNA) autoantibody, antinuclear antibodies (ANA), ATPase autoantibody, and anti-myelin basic protein (MBP) autoantibody.

18. The method of claim 17, wherein the plurality of antibodies comprises IAA, GADA, IA-2A, TGA, TPOA, ThgA, IFNαA and ZnT8A.

19. (canceled)

20. A method of simultaneously determining if a mammal is likely to develop or has developed each one of a plurality of diseases or disorders, wherein each one of the plurality of diseases or disorders is characterized by the presence of at least one autoantibody in a biological sample of the mammal, the method comprising:
(1) contacting the sample with a plurality of antigens, wherein each of the plurality of antigens specifically, and exclusively from each other, binds to one autoantibody associated with one of the plurality of diseases or disorders, wherein each antigen comprises the antigen derivatized with a detectable label (first derivatized antigen) and the antigen derivatized with a tagging label (second derivatized antigen), thus forming a second solution, wherein, if a given autoantibody associated with one of the plurality of diseases or disorders is present in the sample, a corresponding first derivatized antigen-given autoantibody-second derivatized antigen complex is formed;
(2) contacting the second solution with a solid surface comprising a plurality of non-overlapping areas, wherein each one of the plurality of non-overlapping areas is derivatized with a capture molecule that binds specifically to one of the tagging labels, wherein each one of the tagging labels does not bind with more than one of the plurality of non-overlapping areas, thereby immobilizing each of the first derivatized antigen-given autoantibody-second derivatized antigen complexes, if present in the second solution, in a different non-overlapping area (which is thereby known to be associated with the given autoantibody); and
(3) detecting the presence or absence of each one of the first derivatized antigen-given autoantibody-second derivatized antigen complexes by determining presence or absence of the detectable label in the non-overlapping area associated with the given autoantibody;
wherein the presence of a first derivatized antigen-given autoantibody-second derivatized antigen in the non-overlapping area associated with the autoantibody indicates that the mammal is likely to develop or has developed the disease or disorder associated with the autoantibody.

21. The method of claim 20, wherein the disease or disorder comprises an autoimmune disease.

22. The method of claim 20, wherein the disease or disorder comprises at least one selected from the group consisting of acute motor axonal neuropathy (AMAN), Addison's disease, anti-NMDA receptor encephalitis, antiphospholipid syndrome, autoimmune gastritis, autoimmune hepatitis, autoimmune polyendocrine syndrome type 1 (APS-1), celiac disease, choreathetosis, chorea, chronic autoimmune hepatitis, chronic thyroiditis and other auto-immune thyroid diseases, Churg-Strauss syndrome, CREST syndrome, dermatitis herpetiformis, diabetes mellitus type 1 (Type 1 diabetes or T1D), encephalomyelitis, granulomatosis with polyangiitis, Graves' disease, Hashimoto's thyroiditis, inflammatory myopathy, Isaac's Syndrome (autoimmune neuromyotonia), Lambert-Eaton myasthenic syndrome, limbic encephalitis, microscopic polyangiitis, Miller-Fisher Syndrome, Mixed Connective Tissue Disease, multifocal motor neuropathy with conduction block (MMN), multiple sclerosis, myasthenia gravis, neonatal heart block, neuromyelitis optica (Devic's syndrome), opsoclonus myoclonus syndrome, optic neuropathy, paediatric autoimmune neuropsychiatric disease associated with Streptococcus (PANDAS), paraneoplastic cerebellar degeneration, paraneoplastic cerebellar syndrome, polymyositis/dermatomyositis, primary biliary cirrhosis, primary Sjögren's syndrome, rheumatoid arthritis, scleromyositis, scleroderma, Stiff person syndrome, subacute sensory neuronopathy, Sydenham's chorea, systemic lupus erythematosus, systemic sclerosis, and systemic vasculitides.

23. The method of claim 20, wherein the disease or disorder comprises at least one selected from the group consisting of type 1 diabetes, celiac disease, autoimmune thyroiditis and APS-1.

24. (canceled)

25. A kit for determining if a mammal is likely to develop or has developed each one of a plurality of diseases or disorders, wherein each one of the plurality of diseases or disorders is characterized by the presence of at least one autoantibody in a biological sample of the mammal, the kit comprising:
a plurality of antigens, wherein each of the plurality of antigen specifically, and exclusively from each other, binds to one autoantibody associated with one of the plurality of diseases or disorders, wherein each antigen comprises the antigen derivatized with a detectable label (first derivatized antigen) and the antigen derivatized with a tagging label (second derivatized antigen); and
a solid surface comprising a plurality of non-overlapping areas, wherein each one of the plurality of non-overlapping areas is derivatized with a capture molecule that binds with one of the tagging labels, wherein each one of the tagging labels bind to only one of the plurality of non-overlapping areas on the solid surface.

26. The kit of claim 25, wherein the solid substrate surface comprises at least one selected from the group consisting of a silicon wafer, glass, metal, plastic, ceramic, metal alloy, and polymer.
